(19) United States
(12) Patent Application Publication
(10) Pub. No.: US 2025/0049848 A1
(43) Pub. Date: Feb. 13, 2025

(54) METHODS AND COMPOSITIONS FOR ALTERING A TUMOR MICROBIOME

(71) Applicant: BOARD OF REGENTS, THE UNIVERSITY OF TEXAS SYSTEM, Austin, TX (US)

(72) Inventor: Raghu KALLURI, Houston, TX (US)

(73) Assignee: BOARD OF REGENTS, THE UNIVERSITY OF TEXAS SYSTEM, Austin, TX (US)

(21) Appl. No.: 18/719,826

(22) PCT Filed: Dec. 15, 2022
(86) PCT No.: PCT/US2022/081621

§ 371 Date: Jun. 13, 2024

Related U.S. Application Data
(60) Provisional application No. 63/289,805, filed on Dec. 15, 2021.

(51) Int. Cl. A61K 35/17; A61K 39/00; A61K 45/06; A61P 35/00; C07K 16/28; C12Q 1/06; C12Q 1/689

(52) U.S. Cl. CPC A61K 35/17; A61K 39/4613; A61K 39/4631; A61K 39/4644; A61K 45/06; A61P 35/00; C07K 16/2818; C07K 16/2842; C12Q 1/06; C12Q 1/689; A61K 2239/13

(57) ABSTRACT

Aspects of the present disclosure relate to methods and compositions for altering a tumor microbiome. Certain aspects are directed to methods for manipulating a tumor microenvironment for promotion of a tumor restraining microbiome and treatment of cancer. Particular aspects include methods for treating cancer comprising administration of an immunotherapy and an agent capable of disrupting an interaction between α3β1 integrin and α1 homotrimeric type I collagen.

This application claims priority of U.S. Provisional Patent Application No. 63/289,805, filed Dec. 15, 2021, which is hereby incorporated by reference in its entirety.

### BACKGROUND OF THE INVENTION

### I. Field of the Invention

Aspects of the invention relate to at least the fields of cancer biology, microbiology, and medicine.

### II. Background

Recent studies have brought forward an idea that gut and tumor microbiome can play a role in regulating tumor immunity in an impactful manner (1-5). The precise role of the gut and tumor microbiome in the control of cancer continues to be explored. There exists a need for methods and compositions for promoting a tumor-restraining gut and/or tumor microbiome for treatment and maintenance of cancer, as well as for sensitization of a tumor to immunotherapy.

### SUMMARY OF THE INVENTION

The present disclosure fulfills certain needs in the field of cancer medicine by providing methods and compositions for promoting a tumor restraining microbiome by manipulating a tumor microenvironment. Accordingly, disclosed herein are methods for altering a tumor microbiome comprising administering to a tumor microenvironment an effective amount of an agent that is capable of manipulating the tumor microenvironment, for example an agent that disrupts an interaction between α3β1 integrin and α1 homotrimeric type I collagen. In some cases, such an agent is administered in combination with an immunotherapy, for example an immune checkpoint inhibitor.

The present disclosure also includes methods for altering a tumor microbiome, methods for altering a gut microbiome, methods for promoting a tumor restraining microbiome, methods for manipulating a tumor microenvironment, methods for targeting a tumor microenvironment, methods for enhancing response to an immunotherapy, methods for disrupting an interaction between α3β1 integrin and α1 homotrimeric type I collagen, methods for increasing an amount of Campylobacterales bacteria in a tumor microbiome, methods for decreasing an amount of Bacteriodales bacteria in a tumor microbiome, and methods for treating cancer. Methods of the present disclosure can include at least 1, 2, 3, 4, 5, 6 or more of the following steps: administering an agent to a tumor microenvironment, isolating bacteria from a gut microbiome, detecting one or more bacteria in a gut microbiome, isolating bacteria from a tumor microbiome, detecting one or more bacteria from a tumor microbiome, detecting Campylobacterales bacteria from a tumor microbiome, quantifying Campylobacterales bacteria from a tumor microbiome, detecting Bacteriodales bacteria from the tumor microbiome, quantifying Bacteriodales bacteria from the tumor microbiome, administering to a subject an agent that disrupts an interaction between α3β1 integrin and α1 homotrimeric type I collagen, administering to a subject an agent that stimulates angiogenesis in a tumor microenvironment, administering to a subject an agent that inhibits angiogenesis in a tumor microenvironment, administering to a subject an agent that stimulates epithelial to mesenchymal transition in cells of a tumor microenvironment, administering to a subject an agent that inhibits epithelial to mesenchymal transition in cells of a tumor microenvironment, administering to a subject an agent that stimulates pericyte proliferation in a tumor microenvironment, administering to a subject an agent that inhibits pericyte proliferation in a tumor microenvironment, administering to a subject an agent that stimulates cellular metabolism in a tumor microenvironment, administering to a subject an agent that inhibits cellular metabolism in a tumor microenvironment, administering to a subject an agent that activates fibroblast proliferation in a tumor microenvironment, administering to a subject an agent that inhibits fibroblast proliferation in a tumor microenvironment, administering to a subject an agent that degrades extracellular matrix in a tumor microenvironment, administering to a subject an agent that disrupts the lymphatic system, administering to a subject an agent that stimulates lymphangiogenesis, administering to a subject an agent that inhibits lymphangiogenesis, administering to a subject an agent that attenuates hypoxia status, administering to a subject an agent that enhances hypoxia status, administering to a subject an agent that modifies macrophage polarization, administering to a subject an agent that modifies macrophage differentiation, administering a cancer therapy to a subject, administering an immunotherapy to a subject, and administering an immune checkpoint inhibitor to a subject.

Disclosed herein is a method for altering a tumor microbiome comprising administering to a tumor microenvironment an effective amount of an agent that disrupts an interaction between α3β1 integrin and α1 homotrimeric type I collagen. Also disclosed herein is a method of treating a subject for pancreatic cancer, the method comprising: (a) isolating bacteria from the tumor microbiome; and (b) administering to the subject an effective amount of: (i) an agent that disrupts an interaction between α3β1 integrin and α1 homotrimeric type I collagen; and (ii) an immunotherapy. (a) may be performed prior to (b). (a) may be performed subsequent to (b). The immunotherapy may comprise a checkpoint blockade therapy. The checkpoint blockade therapy may comprise an anti-PD-1 antibody. The checkpoint blockade therapy may comprise an anti-CTLA4 antibody.

The agent may be an antibody or an antibody fragment that binds to α3β1 integrin. The agent may be a chimeric antigen receptor (CAR) polypeptide comprising an antigen binding domain that binds to α3β1 integrin. The agent may comprise a cell comprising a nucleic acid encoding a CAR polypeptide, wherein the CAR polypeptide comprises an antigen binding domain that binds to α1 homotrimeric type I collagen. The agent may be an antibody or an antibody fragment that binds to α3β1 integrin. The agent may be a CAR polypeptide comprising an antigen binding domain that binds to α1 homotrimeric type I collagen. The agent may comprise a cell comprising a nucleic acid encoding a CAR polypeptide, wherein the CAR polypeptide comprises an antigen binding domain that binds to α1 homotrimeric type I collagen. The cell may be further defined as one or more of a T cell, a natural killer (NK) cell, an immune cell, an invariant natural killer T (iNKT) cell, a CD4+ T cell, or a CD8+ T cell. The cell may be a NK cell.

Disclosed is a method for altering a tumor microbiome comprising administering to a tumor microenvironment an effective amount of an agent that stimulates angiogenesis in the tumor microenvironment. Also disclosed is a method for altering a tumor microbiome comprising administering to a tumor microenvironment an effective amount of an agent that inhibits angiogenesis in the tumor microenvironment. Also disclosed is a method for altering a tumor microbiome comprising administering to a tumor microenvironment an effective amount of an agent that stimulates epithelial to mesenchymal transition in cells of the tumor microenvironment. Also disclosed is a method for altering a tumor microbiome comprising administering to a tumor microenvironment an effective amount of an agent that inhibits epithelial to mesenchymal transition in cells of the tumor microenvironment. Also disclosed is a method for altering a tumor microbiome comprising administering to a tumor microenvironment an effective amount of an agent that stimulates pericyte proliferation in the tumor microenvironment. Disclosed is a method for altering a tumor microbiome comprising administering to a tumor microenvironment an effective amount of an agent that inhibits pericyte proliferation in the tumor microenvironment. Disclosed is a method for altering a tumor microbiome comprising administering to a tumor microenvironment an effective amount of an agent that stimulates cellular metabolism in the tumor microenvironment. Disclosed is a method for altering a tumor microbiome comprising administering to a tumor microenvironment an effective amount of an agent that inhibits cellular metabolism in the tumor microenvironment. Disclosed is a method for altering a tumor microbiome comprising administering to a tumor microenvironment an effective amount of an agent that activates fibroblast proliferation in the tumor microenvironment. Disclosed is a method for altering a tumor microbiome comprising administering to a tumor microenvironment an effective amount of an agent that inhibits fibroblast proliferation in the tumor microenvironment. Disclosed is a method for altering a tumor microbiome comprising administering to a tumor microenvironment an effective amount of an agent that degrades extracellular matrix in the tumor microenvironment. The agent may be a protease. The protease may be a matrix metalloproteinase. Disclosed is a method for altering a tumor microbiome comprising administering to a tumor microenvironment an effective amount of an agent that stimulates cell-cell adhesion in the tumor microenvironment. Disclosed is a method for altering a tumor microbiome comprising administering to a tumor microenvironment an effective amount of an agent that inhibits cell-cell adhesion in the tumor microenvironment. Disclosed is a method for altering a tumor microbiome comprising administering to a tumor microenvironment an effective amount of an agent that stimulates stromal cell signaling in the tumor microenvironment. Disclosed is a method for altering a tumor microbiome comprising administering to a tumor microenvironment an effective amount of an agent that inhibits stromal cell signaling in the tumor microenvironment. Disclosed is a method for altering a tumor microbiome comprising administering to a tumor microenvironment an effective amount of an agent that disrupts the lymphatic system. Disclosed is a method for altering a tumor microbiome comprising administering to a tumor microenvironment an effective amount of an agent that stimulates lymphangiogenesis. Disclosed is a method for altering a tumor microbiome comprising administering to a tumor microenvironment an effective amount of an agent that inhibits lymphangiogenesis. Disclosed is a method for altering a tumor microbiome comprising administering to a tumor microenvironment an effective amount of an agent that attenuates hypoxia status. Disclosed is a method for altering a tumor microbiome comprising administering to a tumor microenvironment an effective amount of an agent that enhances hypoxia status. Disclosed is a method for altering a tumor microbiome comprising administering to a tumor microenvironment an effective amount of an agent that modifies macrophage polarization. Disclosed is a method for altering a tumor microbiome comprising administering to a tumor microenvironment an effective amount of an agent that modifies macrophage differentiation.

The agent may be one that is effective to increase an amount of Campylobacterales bacteria in the tumor microbiome. The agent may be one that is effective to decrease an amount of Bacteriodales bacteria in the tumor microbiome. The method may further comprise administering to the tumor microenvironment an immunotherapeutic. The immunotherapeutic may comprise a checkpoint blockade therapy. The checkpoint blockade therapy may comprise an anti-PD-1 antibody. The checkpoint blockade therapy may comprise an anti-CTLA4 antibody. The tumor microenvironment may be a pancreatic tumor microenvironment. The method may further comprise administering an antibiotic to the tumor microenvironment. The method may further comprise isolating bacteria from the tumor microbiome. The bacteria may be isolated from the tumor microbiome prior to administering the agent. The bacteria may be isolated from the tumor microbiome after administering the agent. The method may further comprise detecting Campylobacterales bacteria in the tumor microbiome. Detecting Campylobacterales bacteria may comprise sequencing nucleic acid from the tumor microbiome. The Campylobacterales bacteria may be detected prior to administering the agent. The Campylobacterales bacteria may be detected after administering the agent. The method may further comprise detecting Bacteriodales bacteria in the tumor microbiome. Detecting Bacteriodales bacteria may comprise sequencing nucleic acid from the tumor microbiome. The Bacteriodales bacteria may be detected prior to administering the agent. The Bacteriodales bacteria may be detected after administering the agent.

Throughout this application, the term “about” is used to indicate that a value includes the inherent variation of error for the measurement or quantitation method.

The use of the word “a” or “an” when used in conjunction with the term “comprising” may mean “one.” but it is also consistent with the meaning of “one or more,” “at least one.” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Individual,” “subject,” and “patient” are used interchangeably and can refer to a human or non-human.

The compositions and methods for their use can “comprise,” “consist essentially of.” or “consist of”' any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H show results demonstrating that Col1 homotrimer deletion in cancer cells confers beneficial tumor microbiome and immune landscape. FIG. 1A shows bacterial 16S rRNA gene sequencing analysis of intratumoral microbiome and gut (fecal) microbiome from KPPC mice, KPPC;Col1^{pdxKO} mice, or wild-type (tumor-free) littermate control mice. Sequences were classified by taxonomy at the Order level. FIG. 1B shows representative images of hypoxyprobe/pimonidazole immunohistochemistry staining and positivity quantification of tumor sections from KPPC and KPPC;Col1^{pdxKO} mice (n=5 per group). Scale bar: 100 μm. **P<0.01. FIG. 1C shows the evaluation of total bacterial DNA content in pancreatic tissue samples and fecal samples by 16S rRNA gene qRT-PCR analysis. Pancreatic tissue (as pancreatic tumor or normal pancreas) samples and fecal samples were collected from KPPC mice and healthy (tumor-free) littermates, treated either with broad-spectrum antibiotics or vehicle. FIG. 1D shows immune profiling assay of CD11b⁺Gr1⁺ myeloid cells, CD3⁺ T cells, CD4⁺/CD3⁺ T cells, and CD8⁺/CD3⁺ T cells in tumors of KPPC mice and KPPC;Col1^{pdxKO} mice with or without antibiotics treatment (n=4 per group). FIG. 1E shows bacterial 16S rRNA gene sequencing analysis of fecal samples (gut microbiome) from KPPC mice, KPPC;Col1^{pdxKO} mice, or wild-type (tumor-free) littermate control mice with broad-spectrum antibiotics (ABX) treatment. Sequences were classified by taxonomy at the Order level. FIG. 1F shows the overall survival of KPPC mice (n=20) and KPPC;Col1^{pdxKO} mice (n=28) with broad-spectrum antibiotics treatment, compared with untreated KPPC;Col1^{pdxKO} mice. FIGS. 1G and 1H show significantly upregulated expression of interferon response pathway genes (FIG. 1G) in KPPC;Col1^{pdxKO} tumors (n=5) compared to KPPC tumors (n=4), based on bulk RNA sequencing (RNA-seq) data. GSEA revealing significantly upregulated interferon pathways was shown in (FIG. 1H). *P<0.05. **P<0.01. ****P<0.0001. NS: not significant.

FIGS. 2A-2F show bacterial 16S rRNA gene sequencing analysis of intratumoral microbiome composition from KPPC mice and KPPC;Col1^{pdxKO} mice. Sequences were classified by taxonomy at the Phylum (FIG. 2A), Class (FIG. 2B), Order (FIG. 2C), Family (FIG. 2D), Genus (FIG. 2E), and Species levels (FIG. 2F).

FIGS. 3A-3G show bacterial 16S rRNA gene sequencing analysis of gut microbiome composition from fecal samples of KPPC mice and KPPC;Col1^{pdxKO} mice. Sequences were classified by taxonomy at the Phylum (FIG. 3A), Class (FIG. 3B), Order (FIG. 3C), Family (FIG. 3D), Genus (FIG. 3E), and Species (FIG. 3F) levels. (FIG. 3G) shows gene set enrichment analysis (GSEA) revealing significantly upregulated oxidative phosphorylation (OXPHOS) and angiogenesis pathways in KPPC;Col1^{pdxKO} tumors (n=5) compared to KPPC tumors (n=4) from age-matched (53 days) mice, based on bulk RNA-seq analysis.

FIGS. 4A-4G show bacterial 16S rRNA gene sequencing analysis of gut microbiome composition from fecal samples of KPPC mice and KPPC;Col1^{pdxKO} mice that were treated with broad-spectrum antibiotics (ABX). Sequences were classified by taxonomy at the Phylum (FIG. 4A), Class (FIG. 4B), Order (FIG. 4C), Family (FIG. 4D), Genus (FIG. 4E), and Species (FIG. 4F) levels. (FIG. 4G) shows the comparison of overall survival between broad-spectrum antibiotics treated KPPC mice (n=20) and untreated KPPC mice (n=27). NS: not significant.

### DETAILED DESCRIPTION OF THE INVENTION

The present disclosure is based, at least in part, on the surprising discovery that manipulating a tumor microenvironment can lead to conversion of a cancer-promoting microbiome to a tumor-restraining microbiome (e.g., increase in Campylobacterales bacteria; decrease in Bacteriodales bacteria), thereby enhancing an immune response to cancer and sensitizing cancer to immunotherapy. As disclosed herein, as one example, disrupting an interaction between α3β1 integrin and α1 homotrimeric type I collagen can alter a tumor microbiome to a more tumor-restraining makeup, thereby treating a subject for cancer (e.g., pancreatic cancer such as PDAC). Also disclosed are therapeutic methods comprising administering an immunotherapy (e.g., checkpoint blockade therapy) and an agent that modifies a tumor microenvironment (e.g., an agent that disrupts an interaction between α3β1 integrin and α1 homotrimeric type I collagen), thereby altering a tumor microbiome. Further described are various methods for modifying a tumor microbiome comprising manipulating a tumor environment (e.g., angiogenesis, epithelial to mesenchymal transition program, pericytes, metabolism, fibroblasts, extracellular matrix, cell adhesion, stromal signaling, etc.).

### I. Manipulating a Tumor Microenvironment to Alter a Microbiome

Aspects of the present disclosure are directed to methods and compositions for altering a microbiome (e.g., gut microbiome or tumor microbiome) of a subject by manipulating a tumor microenvironment. “Altering a microbiome” describes any change in a makeup of a microbiome, such as an increase or decrease in one or more bacteria types (e.g., species, genera, etc.) or an increase or decrease in total amount of bacteria in a microbiome. Certain aspects of a tumor microbiome are described in, for example, U.S. Patent Application Publication No. US 2020/0129569 A1, incorporated herein by reference in its entirety.

“Manipulating a tumor microenvironment” describes making any change in a makeup of a tumor microenvironment, including changes in the composition, structure, or function of the tumor microenvironment. Such changes include, but are not limited to, increased or decreased vascularization, increased or decreased extracellular matrix, changes in the types of extracellular matrix proteins (e.g., collagens, fibronectin, tenascin, elastins, laminin, etc.), increase or decrease in strength of signaling from the tumor microenvironment (e.g., collagen/integrin signaling such as α1 homotrimeric type I collagen/α3β1 integrin signaling, fibronectin/integrin signaling, laminin/integrin signaling, etc.), and increased or decreased numbers of one or more types of cells in the tumor microenvironment (e.g., stromal cells, fibroblasts, endothelial cells, immune cells, pericytes, etc.).

A tumor microenvironment includes cells, soluble factors, signaling molecules, extracellular matrix components, blood vessels, and other components surrounding and impacting a tumor. Aspects of the tumor microenvironment are described in, for example, Hinshaw D C, Shevde L A. Cancer Res. 2019;79(18):4557-4566 and Hanash S, Schlickelman M. Genome Med. 2014;6(2):12. Published 2014 Feb. 27, incorporated herein by reference in their entirety. Agents targeting any one or more of these tumor microenvironment components are contemplated herein for use in compositions and methods for altering a gut and/or tumor microbiome. Such agents include, but are not limited to, agents that disrupt an interaction between an integrin protein and a fibronectin protein, agents that disrupt an interaction between an integrin protein and a laminin protein, agents that disrupt an interaction between an integrin protein and a collagen protein, agents that disrupt an interaction between an α3β1 integrin and al homotrimeric type I collagen, agents that stimulate angiogenesis, agents that inhibit angiogenesis, agents that stimulate epithelial to mesenchymal transition in cells of the tumor microenvironment, agents that inhibit epithelial to mesenchymal transition in cells of the tumor microenvironment, agents that stimulate pericyte proliferation, agents that inhibit pericyte proliferation, agents that stimulate cellular metabolism, agents that inhibit cellular metabolism, agents that activate fibroblast proliferation, agents that inhibit fibroblast proliferation, agents that degrade extracellular matrix (e.g., matrix metalloproteinases or other enzymes), agents that stimulate cell-cell adhesion, agents that inhibit cell-cell adhesion, agents that stimulate stromal cell signaling, agents that inhibit stromal cell signaling in the tumor microenvironment, agents that disrupt the lymphatic system, agents that stimulate lymphangiogenesis, agents that inhibit lymphangiogenesis, agents that attenuate hypoxia status, agents that enhance hypoxia status, agents that modify macrophage polarization, and agents that modify macrophage differentiation. Example types of agents targeting a tumor microenvironment include antibodies (e.g., anti-α3β1 integrin antibodies, anti-al homotrimeric type I collagen antibodies), antibody fragments, antibody-like molecules, polypeptides, oligonucleotides (e.g., antisense oligonucleotides, siRNAs, etc.), chimeric antigen receptors (CARs), CAR T cells, CAR NK cells, and small molecules (e.g., integrin-binding molecules). Certain example agents which may be useful in targeting a tumor microenvironment are described in PCT Patent Application Publication Nos. WO 2020/257296 and WO 2020/081714, incorporated herein by reference in their entirety.

As used herein, an agent that “disrupts an interaction” between two molecules is capable of reducing a binding interaction between the two molecules. For example, an agent that disrupts an interaction between α3β1 integrin and α1 homotrimeric type I collagen is capable of reducing the binding between these two molecules, thereby reducing resultant signaling. The ability of an agent to disrupt an interaction between two molecules may be readily detected and measured by various means recognized in the art including, for example, various binding assays such as ELISA. Certain example binding assays contemplated herein include those described in Pollard T D. Mol Biol Cell. 2010;21(23):4061-4067 and Hunter S A, Cochran J R. Methods Enzymol. 2016;580:21-44, incorporated herein by reference in their entirety. An ability of an agent to disrupt an interaction may also be measured by, for example, measuring a decrease in cell signaling that results from interaction of two molecules such as a cell surface receptor and a ligand.

### II. Cancer Therapy

In some aspects, the disclosed methods comprise administering a cancer therapy to a subject or patient. The cancer therapy may be chosen based on an expression level measurements, alone or in combination with the clinical risk score calculated for the subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, disclosed are methods for treating cancer originating from the pancreas. In some aspects, disclosed are methods for treating pancreatic ductal adenocarcinoma (PDAC).

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### A. Radiotherapy

In some aspects, a radiotherapy, such as ionizing radiation, is administered to a subject. As used herein, “ionizing radiation” means radiation comprising particles or photons that have sufficient energy or can produce sufficient energy via nuclear interactions to produce ionization (gain or loss of electrons). A non-limiting example of ionizing radiation is x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97. 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### B. Cancer Immunotherapy

In some aspects, the methods comprise administration of a cancer immunotherapy. Cancer immunotherapy (sometimes called immuno-oncology, abbreviated IO) is the use of the immune system to treat cancer. Immunotherapies can, in some cases, be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and certain examples are described below.

### 1. Checkpoint Inhibitors and Combination Treatment

Aspects of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT,” “checkpoint blockade immunotherapy,” “checkpoint blockade therapy,” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3′s main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tel cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. No. 9,605,070, U.S. Pat. No. 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another aspect, the antibody has at least about 70, 75, 80, 85, 90, 95, 97, or 99% (or any derivable range or value therein) variable region amino acid sequence identity with the above-mentioned antibodies.

### 2. Activator of Co-Stimulatory Molecules

In some aspects, the immunotherapy comprises an activator (also “agonist”) of a co-stimulatory molecule. In some aspects, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include activating antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### C. Oncolytic Virus

In some aspects, the cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### D. Chemotherapies

In some aspects, a therapy of the present disclosure comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection.

Other suitable chemotherapeutic agents include antimicrotubule agents, e.g., Paclitaxel (“Taxol”) and doxorubicin hydrochloride (“doxorubicin”). Doxorubicin is absorbed poorly and is preferably administered intravenously. In certain aspects, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred in certain cases. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

The amount of the chemotherapeutic agent delivered to a patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### E. Hormone Therapy

In some aspects, a cancer therapy of the present disclosure is a hormone therapy. In particular aspects, a prostate cancer therapy comprises hormone therapy. Various hormone therapies are known in the art and contemplated herein. Examples of hormone therapies include, but are not limited to, luteinizing hormone-releasing hormone (LHRH) analogs, LHRH antagonists, androgen receptor antagonists, and androgen synthesis inhibitors.

### F. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### G. Additional Cancer Therapies

Therapeutic methods disclosed herein may comprise one or more additional cancer therapies. A cancer therapy of the disclosure may comprise, for example, cryoablative therapy, high-intensity ultrasound (also “high-intensity focused ultrasound”), photodynamic therapy, laser ablation, and/or irreversible electroporation. A cancer therapy of the disclosure may comprise 1, 2, 3, 4, 5, or more distinct therapeutic methods.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### III. Pharmaceutical Compositions

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In instances where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. A unit dose may comprise a single administrable dose.

The quantity to be administered, both according to number of treatments and unit dose, depends on the treatment effect desired. An effective dose is understood to refer to an amount necessary to achieve a particular effect. Doses in the range from 10 mg/kg to 200 mg/kg may affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

The therapeutically effective or sufficient amount of the immune checkpoint inhibitor, such as an antibody and/or microbial modulator, that is administered to a human may be in the range of about 0.01 to about 50 mg/kg of patient body weight whether by one or more administrations. The therapy used may be about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. A therapy described herein may be administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

The effective dose of the pharmaceutical composition may be one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). The dose may be one that can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. The therapeutic agent that may be administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Assay Methods

### A. Sequencing

Aspects of the present disclosure include sequencing of nucleic acid from a sample. including a tumor sample, tumor microbiome sample, and/or gut microbiome sample. In some aspects, disclosed are methods comprising sequencing genomic DNA of bacteria from a tumor microbiome or gut microbiome. Such methods may be useful in, for example, detecting and identifying a particular bacteria in a tumor microbiome or gut microbiome. The methods of the disclosure may include a sequencing method. Exemplary sequencing methods include those described below.

### 1. Massively Parallel Signature Sequencing (MPSS)

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

### 2. Polony Sequencing

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

### 3. 454 Pyrosequencing

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

### 4. Illumina (Solexa) Sequencing

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1x coverage per hour per instrument, and one human genome re-sequenced (at approx. 30×) per day per instrument (equipped with a single camera).

### 5. SOLiD Sequencing

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

### 6. Ion Torrent Semiconductor Sequencing

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### 7. DNA Nanoball Sequencing

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

### 8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

### 9. Single Molecule Real Time (SMRT) Sequencing

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### B. Additional Assay Methods

Methods may involve amplifying and/or sequencing one or more target genomic regions using at least one pair of primers specific to the target genomic regions. The primers may be heptamers. Enzymes may be added such as primases or primase/polymerase combination enzyme to the amplification step to synthesize primers.

Arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, ddPCR (“digital droplet PCR” or “droplet digital PCR”), nCounter (nanoString), BEAMing (Beads, Emulsions, Amplifications, and Magnetics) (Inostics), ARMS (Amplification Refractory Mutation Systems), RNA-Seq. TAm-Seg (Tagged-Amplicon deep sequencing), PAP (Pyrophosphorolysis-activation polymerization), next generation RNA sequencing, northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form, although the single-stranded form is preferred.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. The density may be or may be at least 50, 100, 200, 300, 400, 500 or more probes/cm2 (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain aspects may involve the use of arrays or data generated from an array. Data may be readily available. Moreover, an array may be prepared in order to generate data that may then be used in correlation studies.

### V. Antibodies

Aspects of the disclosure relate to antibodies, antigen binding fragments thereof, or polypeptides capable of specifically binding to α3β1 integrin, COL1A1, COL1A2, or any other tumor microenvironment protein. In some aspects, the present disclosure includes antibodies, antigen binding fragments thereof, or polypeptides capable of disrupting an interaction between α3β1 integrin and α1 homotrimeric type I collagen.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, sec, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4:302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bispecific, meaning the two antigen-binding sites have different antigen specificities.

Bispecific antibodies are a class of antibodies that have two paratopes with different binding sites for two or more distinct epitopes. Bispecific antibodies can be biparatopic, wherein a bispecific antibody may specifically recognize a different epitope from the same antigen. Bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

Multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for triabodies, tetrabodies, and higher order antibody multimers. (sec, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1 (or LCDR1), CDR-L2 (or LCDR2), and CDR-L3 (or LCDR3). The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1 (or HCDR1), CDR-H2 (or HCDR2), and CDR-H3 (or HCDR3). The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24:8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

Minimizing the antibody polypeptide sequence from the non-human species may optimize chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhocyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### B. Functional Antibody Fragments and Antigen-Binding Fragments

### 1. Antigen-Binding Fragments

Certain aspects relate to antibody fragments, such as antibody fragments that bind to α3β1 integrin, COL1A1, COL1A2, or any other tumor microenvironment protein. The term functional antibody fragment includes antigen-binding fragments of an antibody that retain the ability to specifically bind to an antigen. These fragments are constituted of various arrangements of the variable region heavy chain (VH) and/or light chain (VL); and may include constant region heavy chain 1 (CH1) and light chain (CL). In some aspects, they lack the Fc region constituted of heavy chain 2 (CH2) and 3 (CH3) domains. Aspects of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino-and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### E. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly (n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### VI. Inhibitory Oligonucleotides

In some aspects, the disclosure relates to inhibitory oligonucleotides that inhibit the gene expression of a protein in a tumor microenvironment, for example an integrin protein or a collagen protein. In some aspects, inhibitory oligonucleotides of the disclosure inhibit expression of α3β1 integrin. In some aspects, inhibitory oligonucleotides of the disclosure inhibit expression of alpha-1 type I collagen (COL1A1). In some aspects, inhibitory oligonucleotides of the disclosure inhibit expression of alpha-2 type I collagen (COL1A2). Examples of an inhibitory oligonucleotides include but are not limited to siRNA (small interfering RNA), short hairpin RNA (shRNA), double-stranded RNA, an antisense oligonucleotide, a ribozyme, and an oligonucleotide encoding any thereof. An inhibitory oligonucleotide may inhibit the transcription of a gene or prevent the translation of a gene transcript in a cell. An inhibitory oligonucleotide acid may be from 16 to 1000 nucleotides long, and in certain embodiments from 18 to 100 nucleotides long. The oligonucleotide may have at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of α3β1 integrin, COL1A1, COL1A2, or other tumor microenvironment protein by at least 10%, 20%, 30%, or 40%, more particularly by at least 50%, 60%, or 70%, and most particularly by at least 75%, 80%, 90%, 95%, 99%, or 100% more or any range or value in between the foregoing.

In further embodiments, there are synthetic oligonucleotides that are α3β1 integrin, COL1A1, and/or COL1A2 inhibitors. An inhibitor may be between 17 to 25 nucleotides in length and comprises a 5′ to 3′ sequence that is at least 90% complementary to the 5′ to 3′ sequence of a mature α3β1 integrin, COL1A1, and/or COL1A2 mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature [POI] mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support-an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides, peptide nucleic acids, or a 5′ (E)-vinyl-phosphonate (VP) modification. In some embodiments, the oligonucleotides has one or more phosphorothioated DNA or RNA bases.

### VII. Administration of Therapeutic Combinations

The therapy provided herein may comprise administration of a combination of therapeutic agents, such as a first cancer therapy (e.g., an agent that disrupts an interaction between α3β1 integrin and α1 homotrimeric type I collagen) and a second cancer therapy (e.g., an immunotherapy). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

In some embodiments, the first cancer therapy and the second cancer therapy are administered substantially simultaneously. In some embodiments, the first cancer therapy and the second cancer therapy are administered sequentially. In some embodiments, the first cancer therapy, the second cancer therapy, and a third cancer therapy (e.g., chemotherapy) are administered sequentially. In some embodiments, the first cancer therapy is administered before administering the second cancer therapy. In some embodiments, the first cancer therapy is administered after administering the second cancer therapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1. 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 M; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87. 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### VIII. Kits

Certain aspects of the present disclosure also concern kits containing compositions of the disclosure or compositions to implement methods disclosed herein. In some embodiments, kits can be used to evaluate one or more biomarkers. In some embodiments, kits can be used to detect one or more type (e.g., species, genera, etc.) of bacteria from a microbiome sample (e.g., gut microbiome, tumor microbiome). In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

### IX. Cells

### A. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### B. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation.

### X. Detecting a Genetic Signature

Particular embodiments concern the methods of detecting a genetic signature in an individual, including in a microbiome (e.g., gut microbiome, tumor microbiome) from an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

### A. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage.

### B. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp. 70 bp. 75 bp, 80 bp, 85 bp, 90 bp, 95 bp. 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

### C. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Deletion of Col1 Homotrimer in Pancreatic Cancer Cells Alters Intratumoral Microbiome and Immune Landscape

### Results

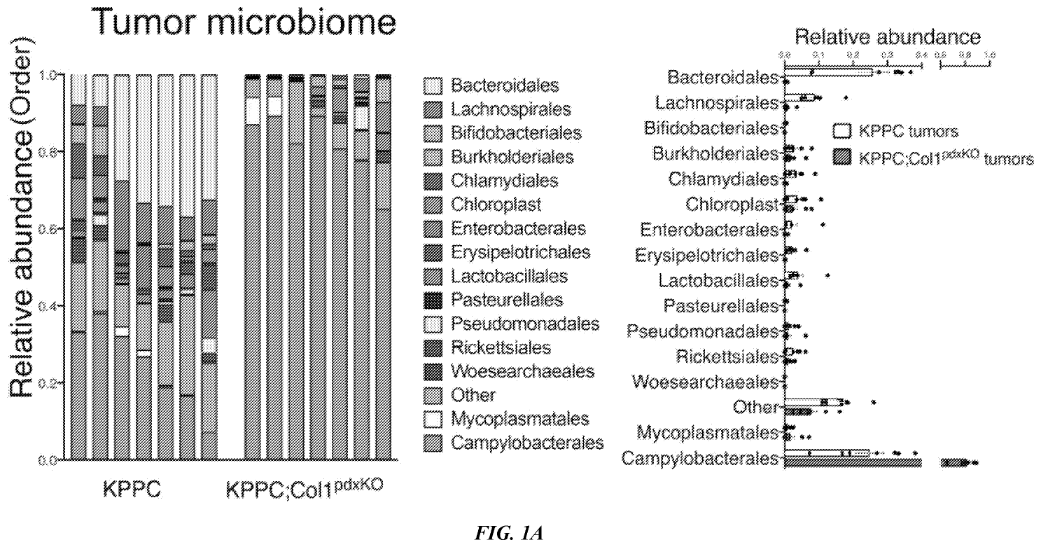

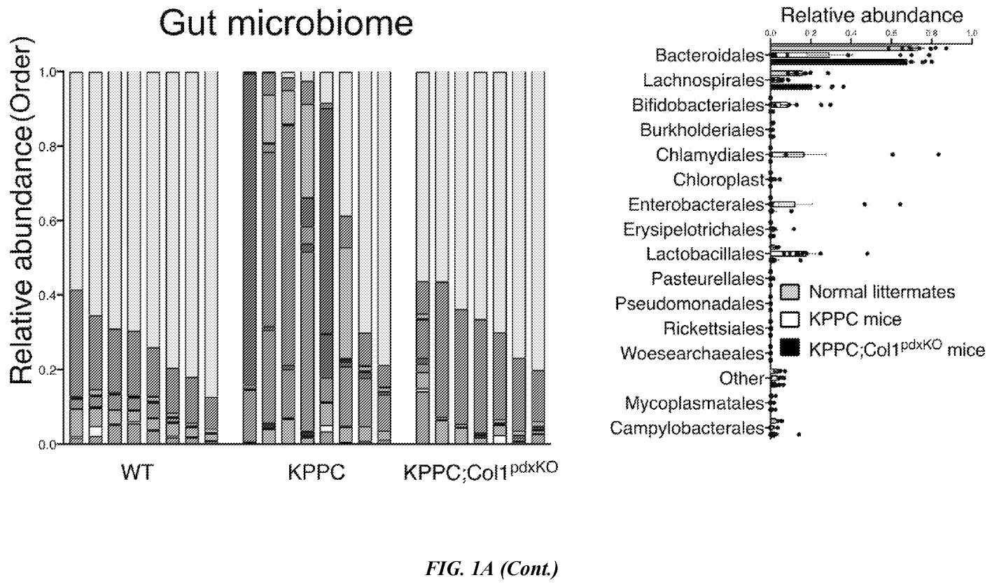

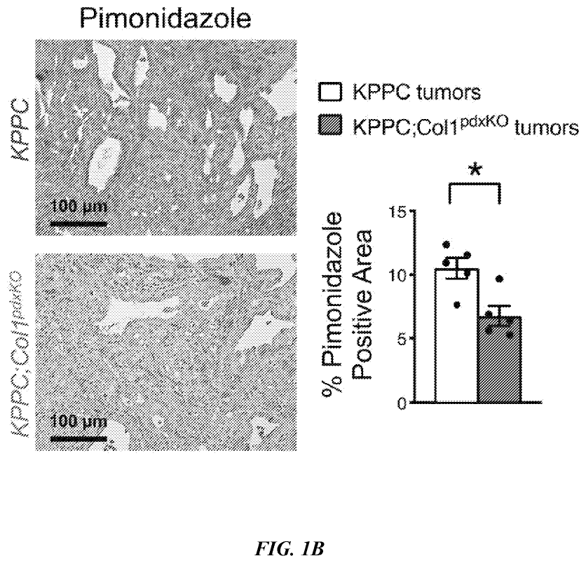

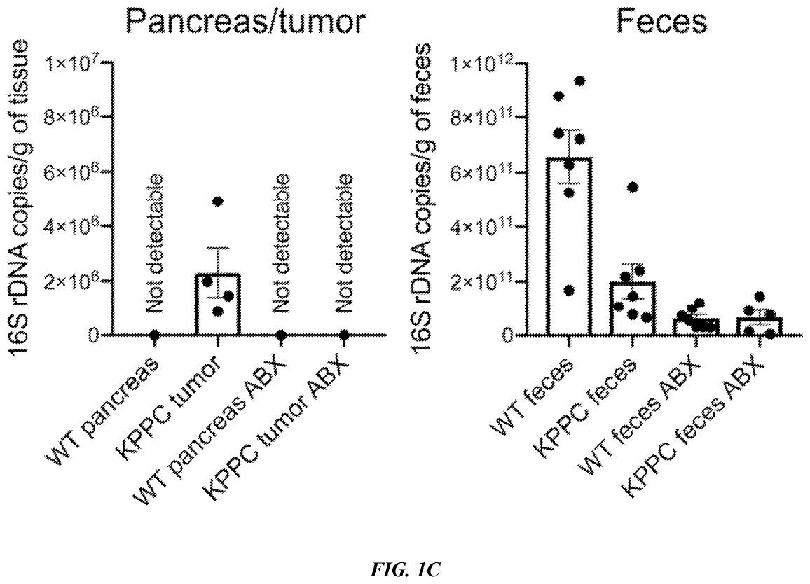

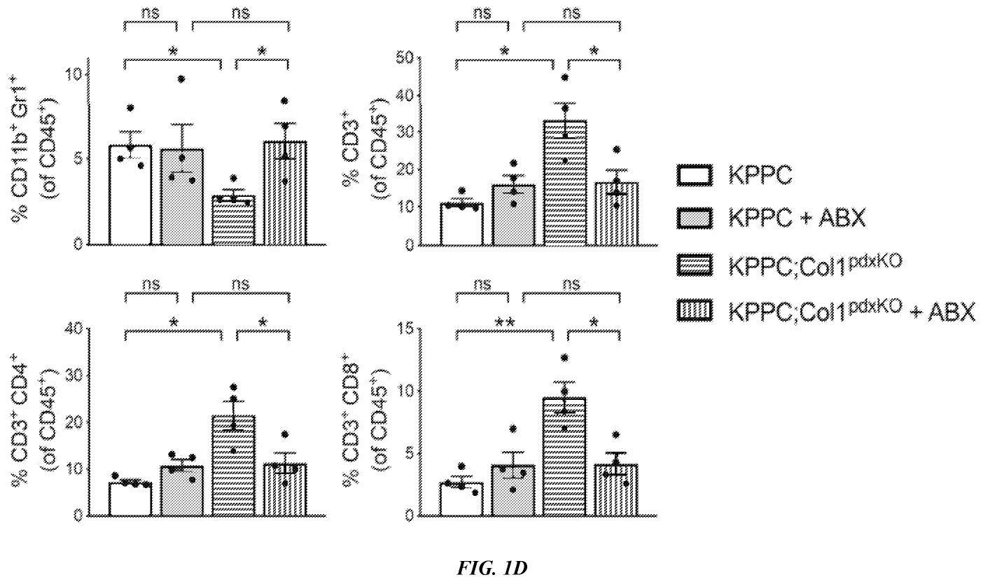

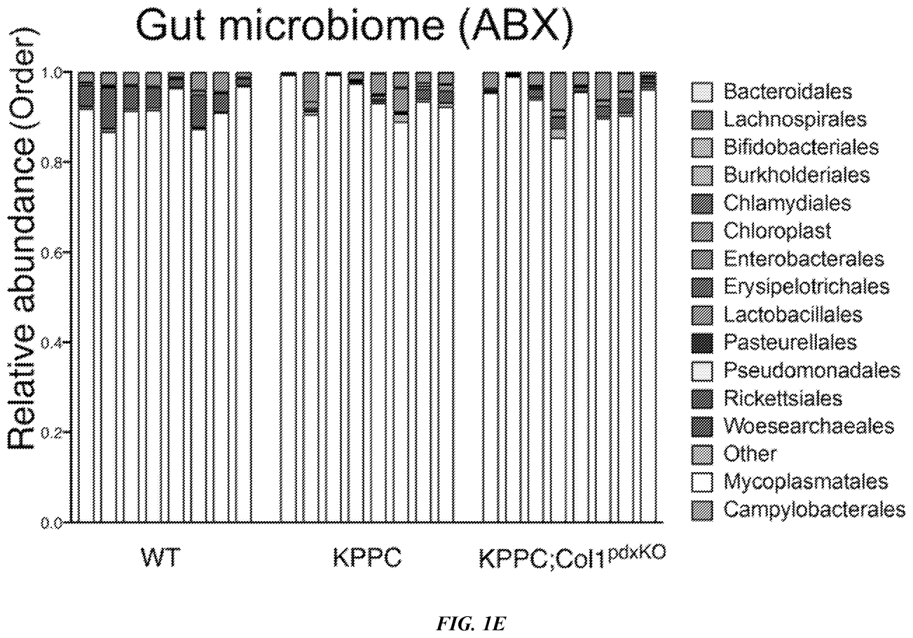

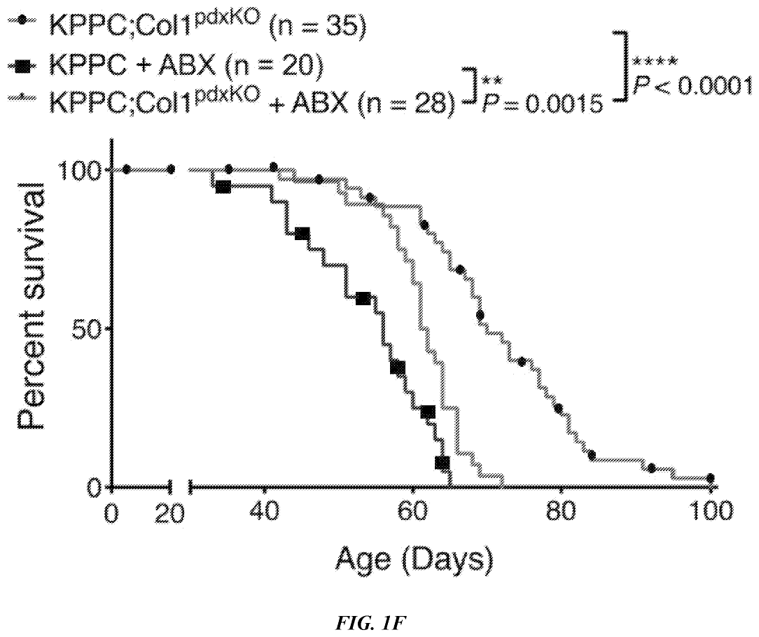

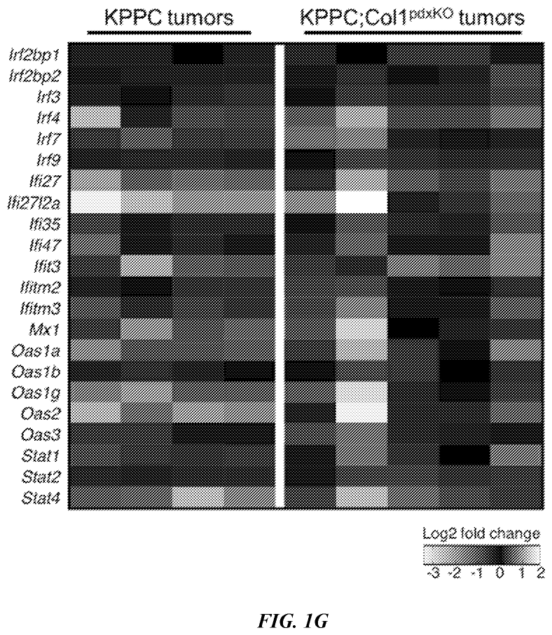

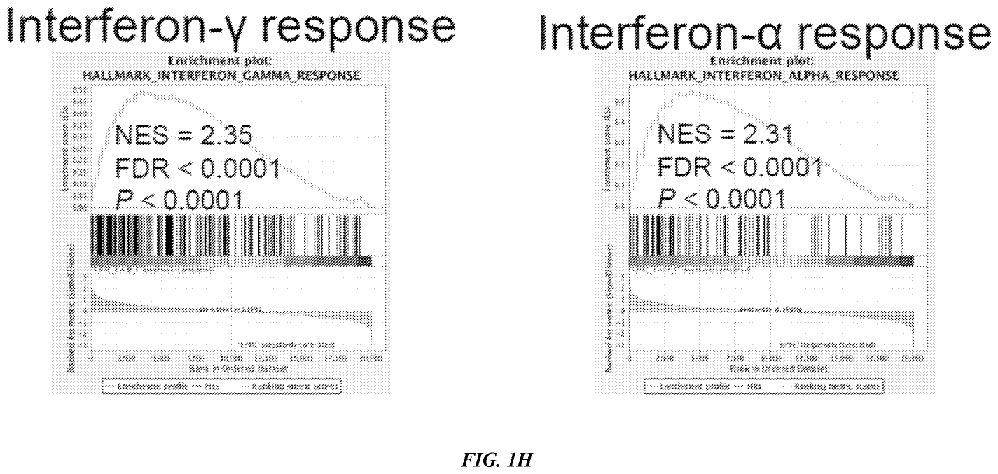

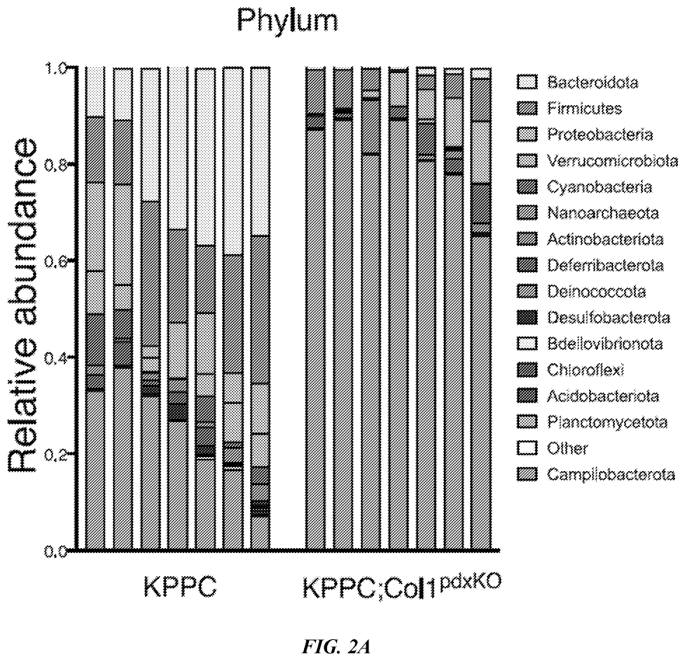

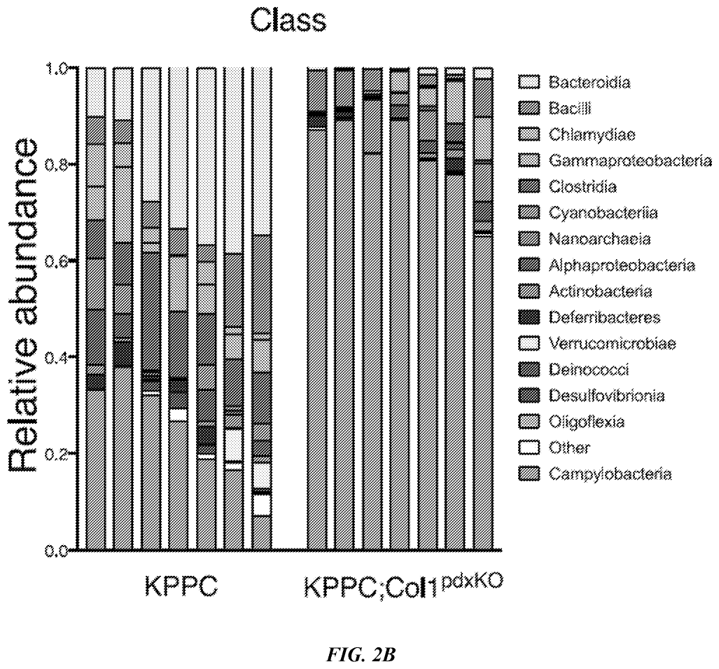

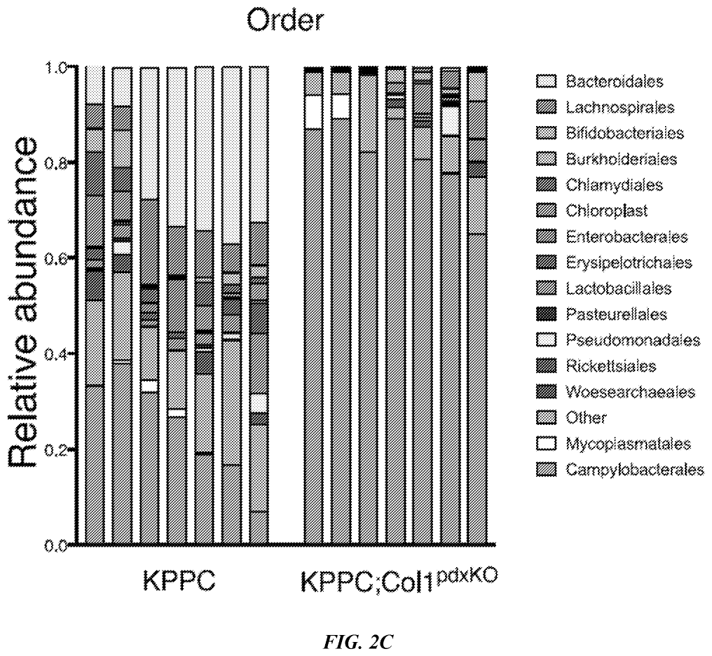

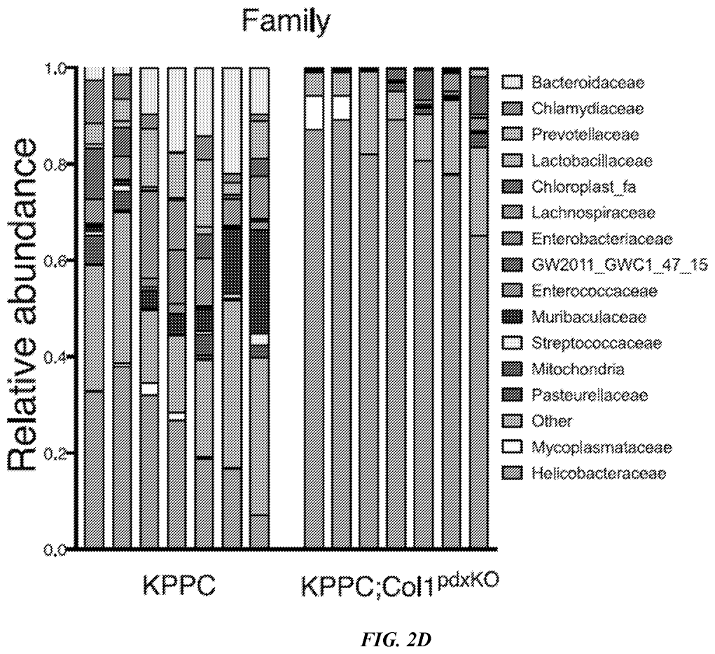

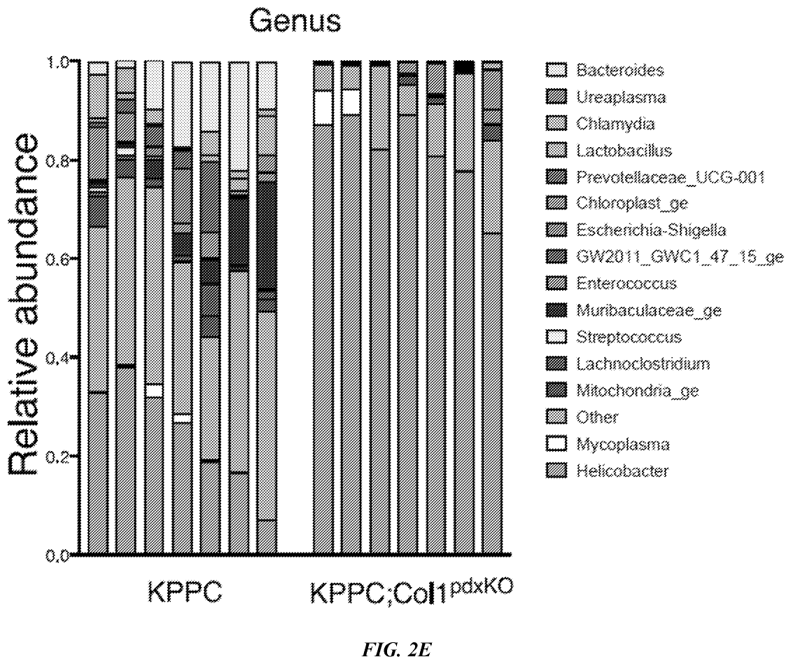

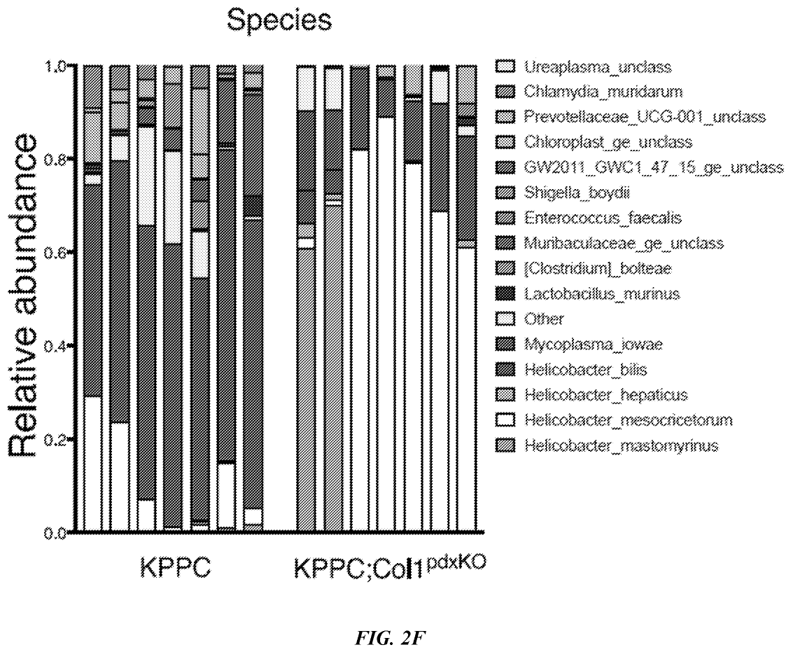

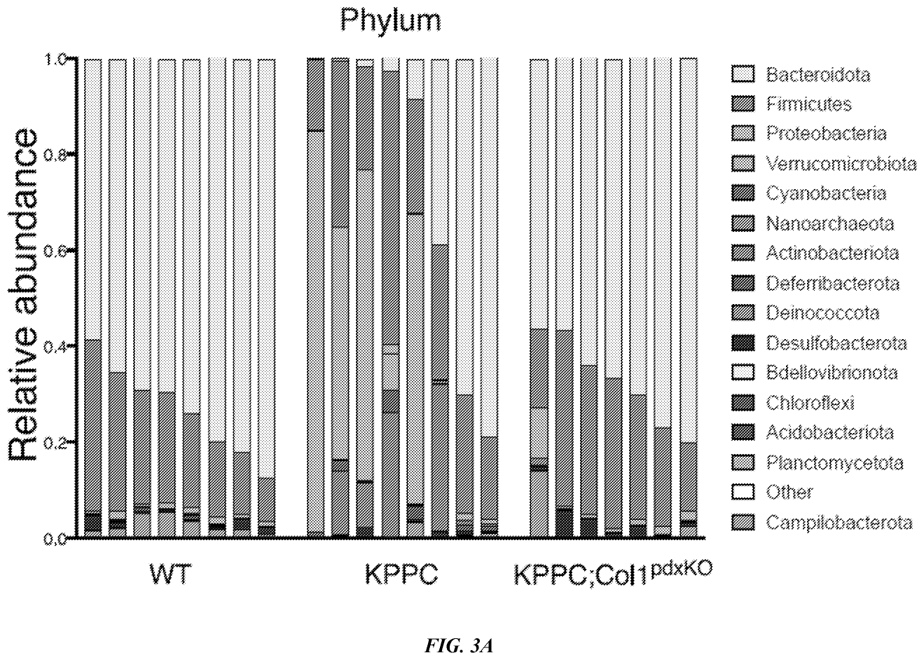

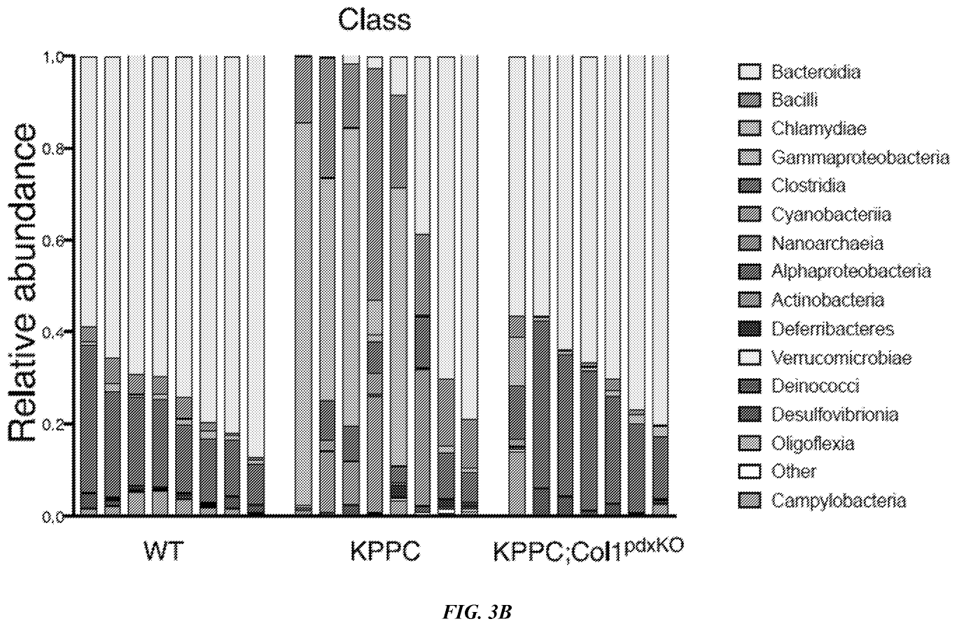

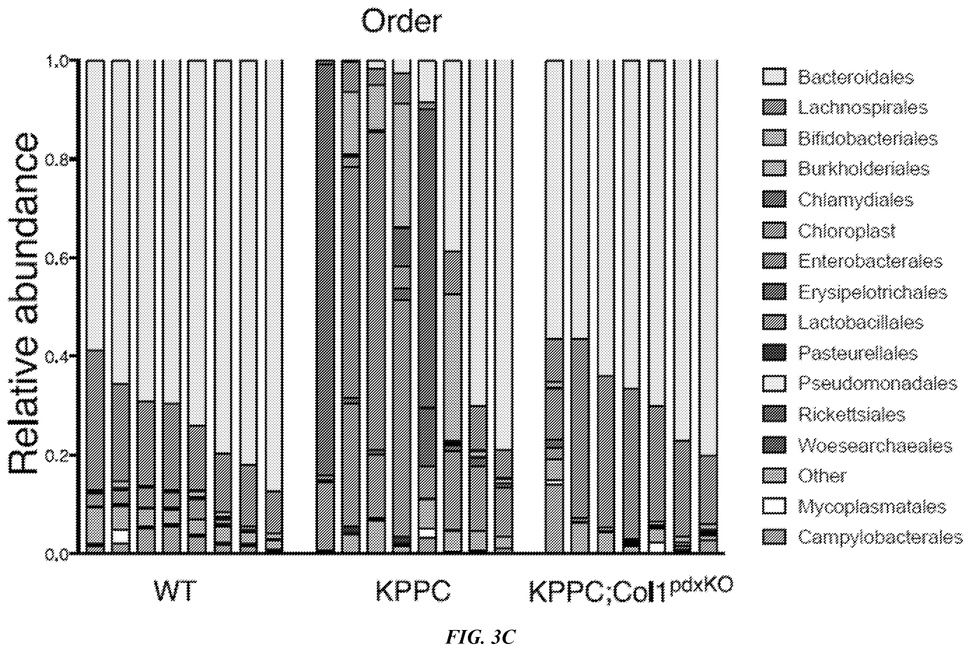

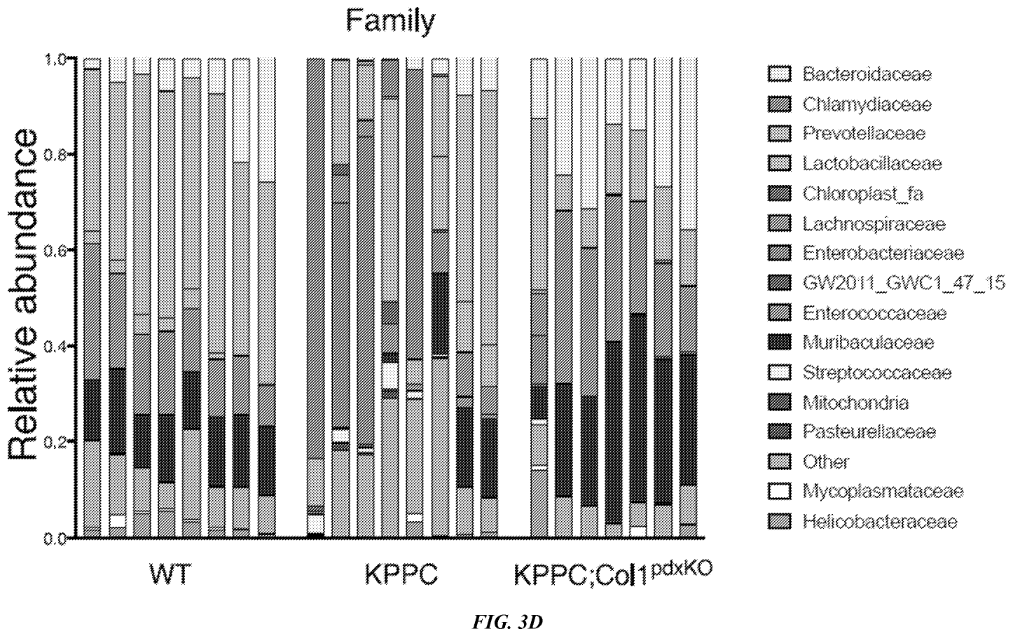

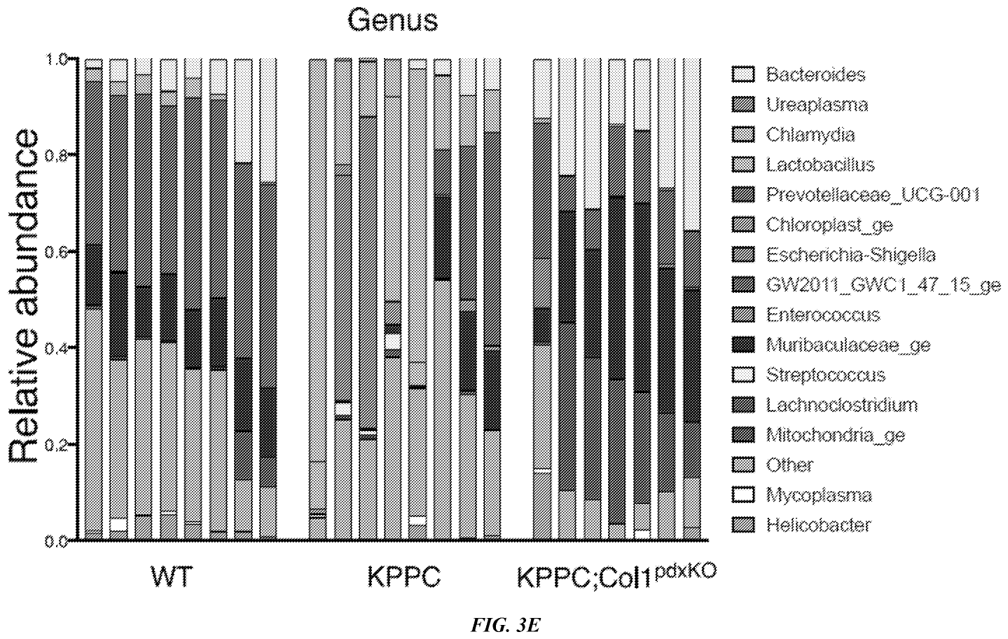

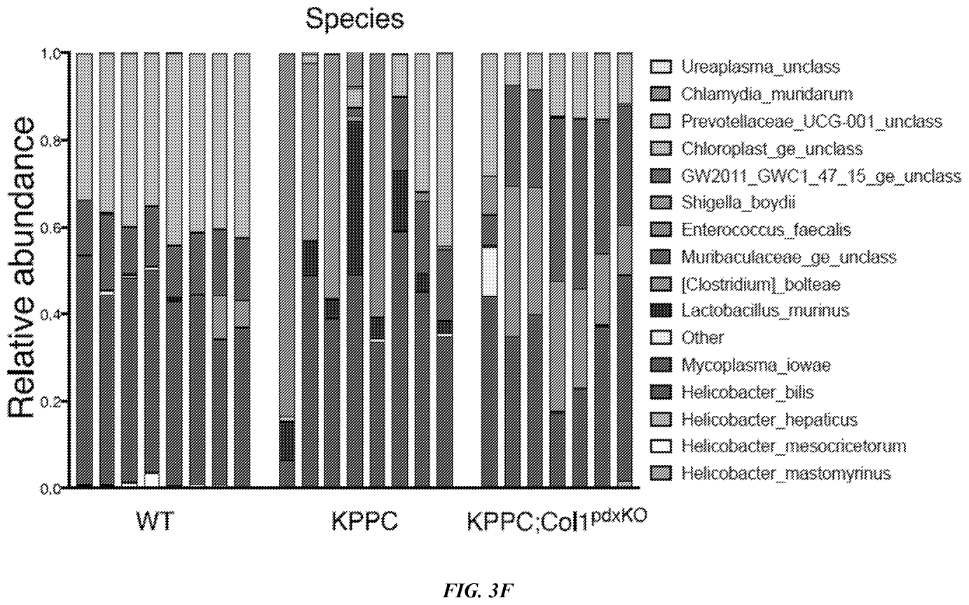

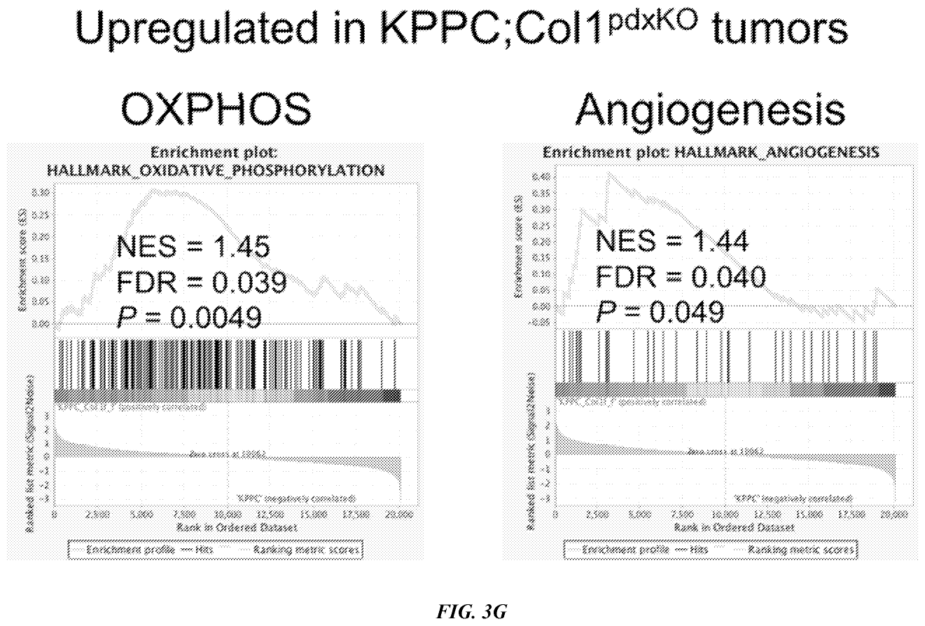

The gut and tumor microbiome was examined in relation to the impact of Col1 deletion on tumor immunity in KPPC;Col1^{pdxKO} tumors. When examined by 16S rRNA gene sequencing analysis, KPPC;Col1^{pdxKO} mice revealed significantly altered intratumoral microbiome profile with decreased Bacteroidales and increased Campylobacterales, as compared with tumors from KPPC control mice (FIG. 1A and FIGS. 2A-2F). Col1 deletion preserved gut microbiome homeostasis of KPPC;Col1^{pdxKO} mice with taxonomic patterns similar to that of tumor-free (WT) littermates. The KPPC control mice exhibited aberrant gut microbiome composition with greater individual variation among different mice (FIG. 1A and FIG. 3A), generally associated with more advanced tumor progression (3). The decreased Bacteroidales (anaerobic) and increased Campylobacterales (microaerophilic) in the tumors were associated with the alleviated intratumoral hypoxia in KPPC;Col1^{pdxKO} tumors compared with KPPC tumors (FIG. 1B). In this regard, bulk RNA sequencing (RNA-seq) on total RNA from tumor tissues of KPPC;Col1^{pdxKO} mice and KPPC mice revealed elevated gene expression levels of oxidative phosphorylation (OXPHOS) and angiogenesis pathways by GSEA in KPPC;Col1 ^{pdxKO} tumors compared with KPPC tumors (FIG. 3B). Normal pancreas from healthy littermates exhibited very low levels of total bacterial DNA content while pancreatic tumors from KPPC mice exhibited detectable bacterial DNA content by quantitative PCR (FIG. 1C), consistent with previous notion that bacterial translocation occurs as pancreatic tumor progresses (3).

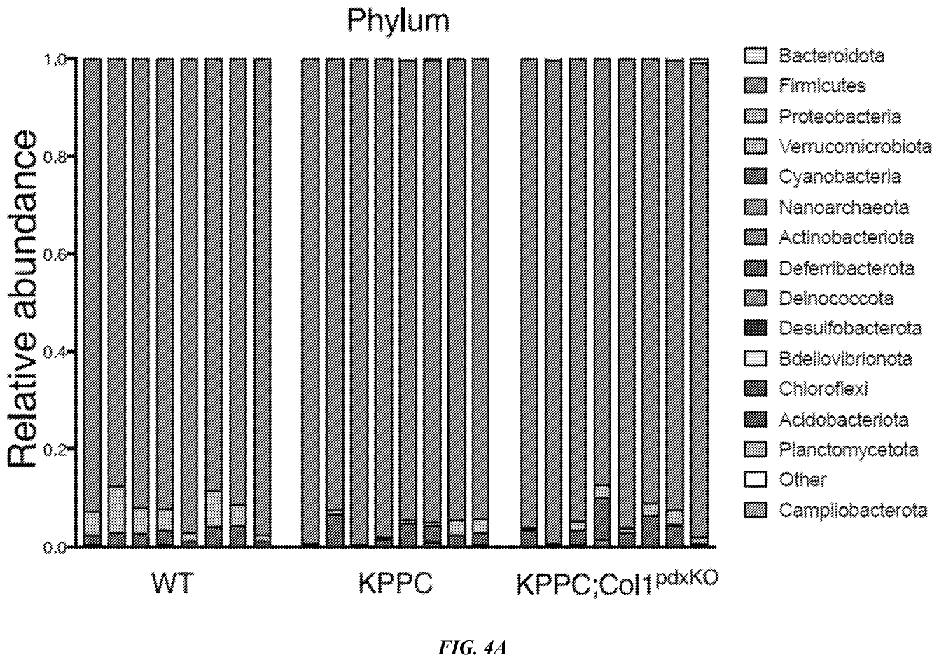

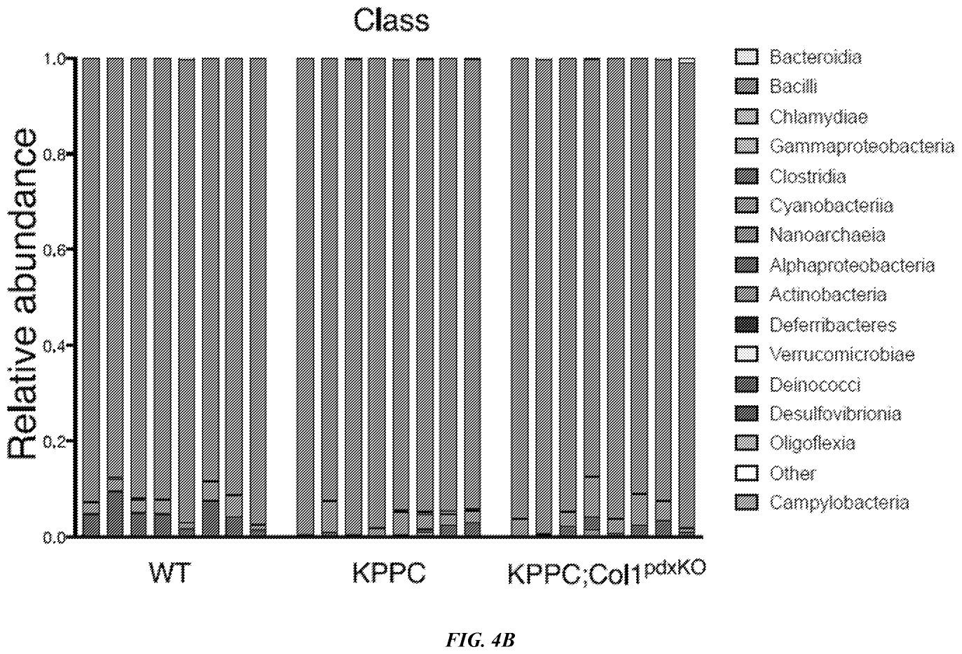

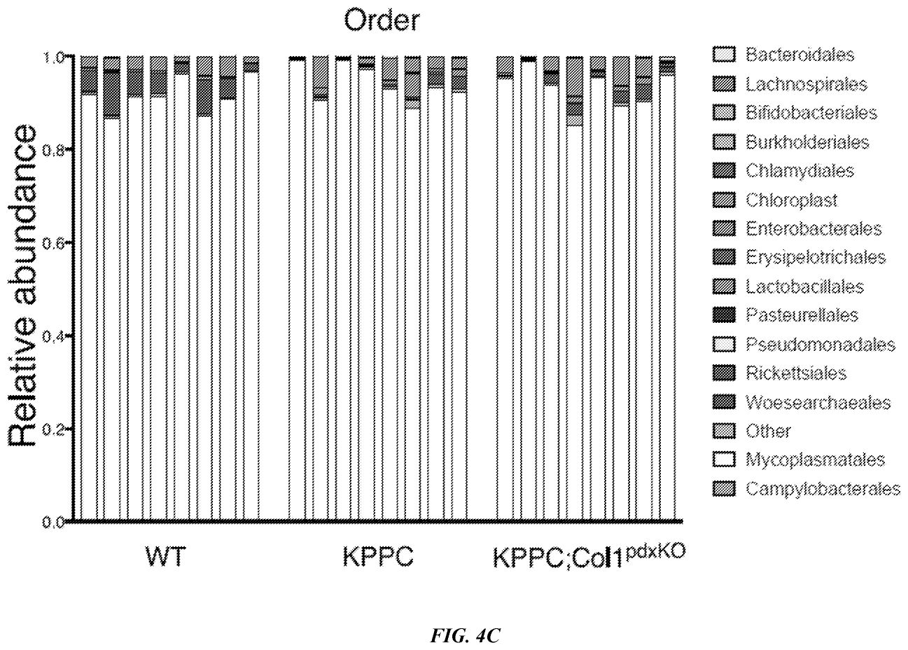

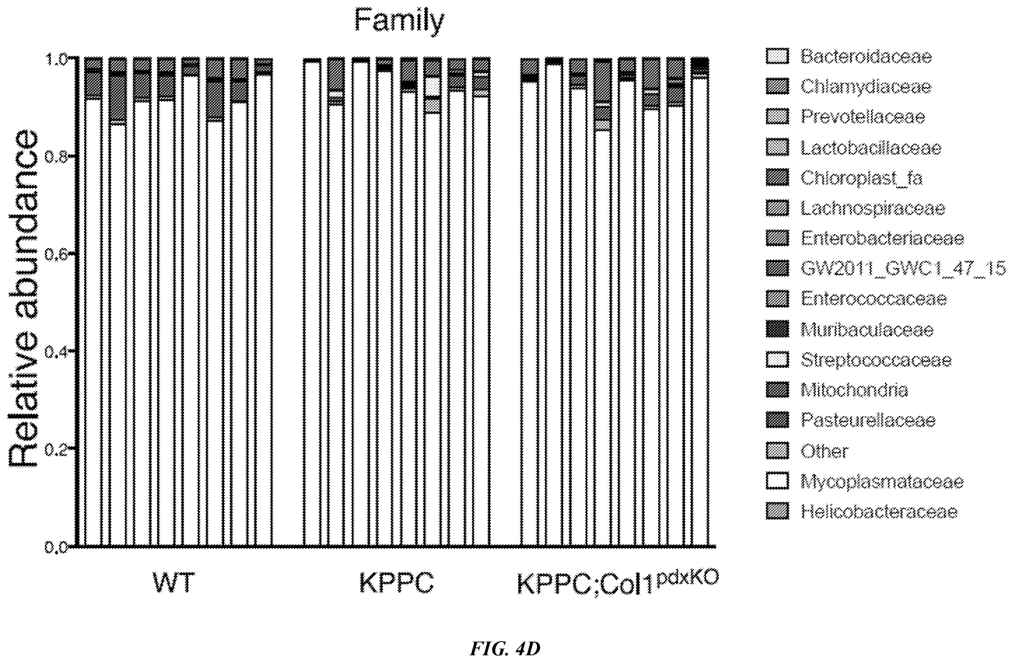

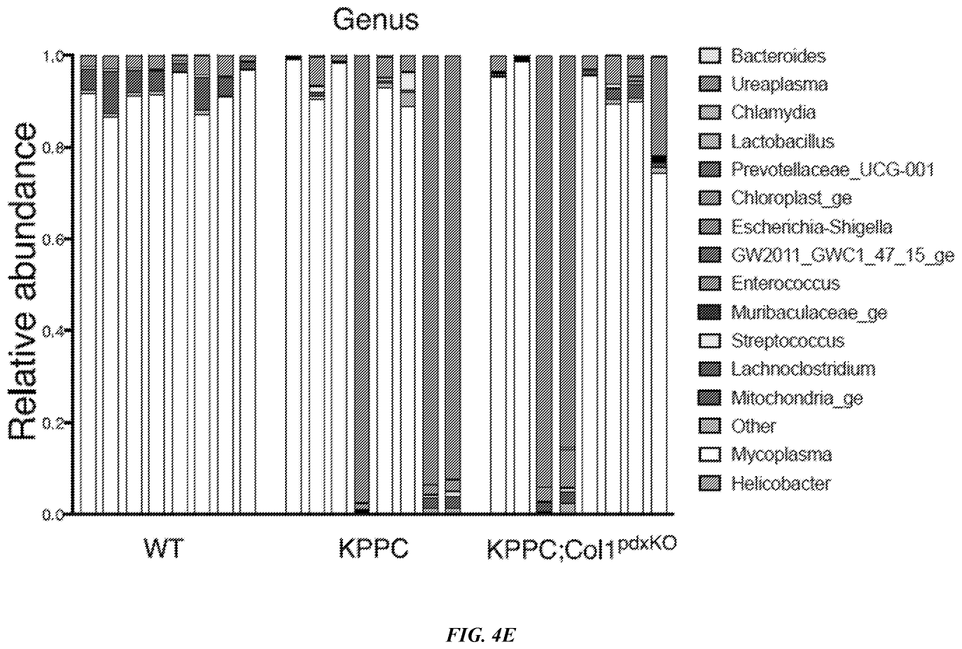

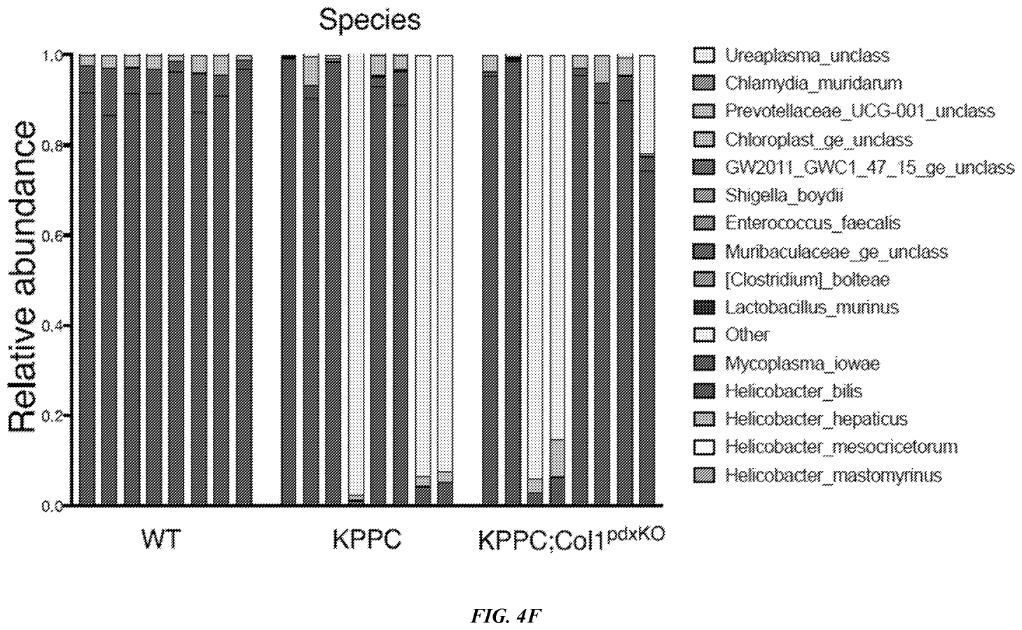

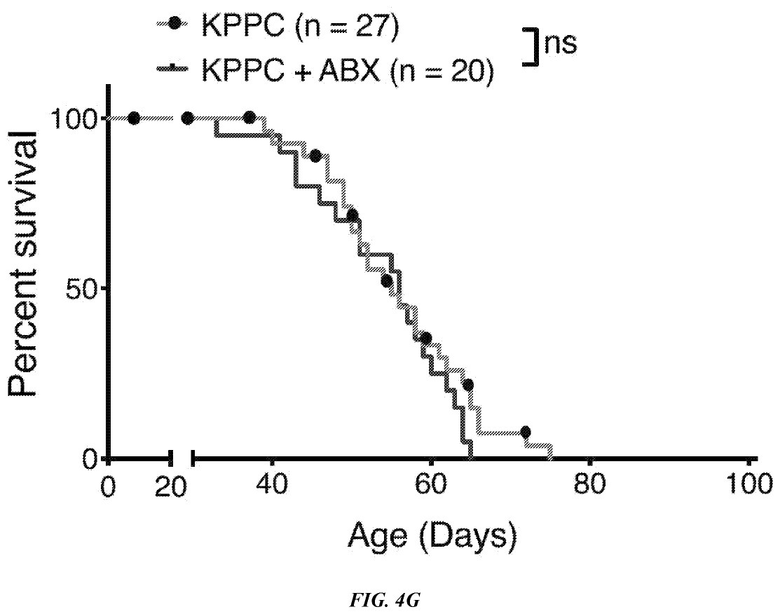

The altered microbiome in KPPC;Col1^{pdxKO} mice was also associated with changes in intratumoral immune cell profile including decreased MDSCs and increased T cells with putative benefit in restraining PDAC (FIG. 1D). In order to directly determine the impact of microbiome on the tumor immunity, a combination of broad-spectrum antibiotics (containing vancomycin, neomycin, metronidazole, and amphotericin) treatment (3) was used to evaluate if gut and tumor microbiome can be altered. As reported before, this combination of antibiotics completely ablated the bacteria in the tumors to undetected levels similar to normal pancreas (FIG. 7C). Similarly, and as expected, antibiotics treatment also reduced the gut microbiome content but did not eliminate it (FIGS. 1C and 1E), likely due to the dramatically abundant microbial content in feces, as reported by others (6,7). Nevertheless, 16S rRNA gene sequencing analysis of the remaining gut microbiome after antibiotics treatment revealed complete loss of the microbial taxonomy difference between KPPC;Col1^{pdxKO} mice and KPPC mice (FIG. 1E and FIG. 4A).

Elimination of the tumor and gut microbiome by antibiotics treatment resulted in altered immune profile in KPPC;Col1^{pdxKO} tumors with increased MDSCs and decreased T cells (FIG. 1D), and resulted in significantly shortened overall survival of KPPC;Col1^{pdxKO} mice (FIG. 1F). Such impact of broad-spectrum antibiotics treatment was not observed in the KPPC mice (FIG. 4B). These results indicated that the overall impact of Col1 homotrimers is partly due to Col1-homotrimer-associated microbiome and the related immune profile acquisition. These results confirm that deletion of Col1 homotrimers production from cancer cells leads to induction of protective microbiome associated with beneficial immune cell profile. Bulk RNA-seq of KPPC;Col1^{pdxKO} tumors further revealed the increased expression profiles of genes related to type I interferon (IFN) pathway (FIGS. 1G and 1H), which is associated with microbiome-regulated antitumor immune responses (1,8).

These studies demonstrated that cancer-derived oncogenic Col1 homotrimers plays an important role in promoting immune suppression. Col1 homotrimer deletion in cancer cells increases T cell infiltration into the tumors and augments the efficacy of anti-PD-1 checkpoint blockade therapy, associated with increased overall survival of mice. Therefore, it appears that Col1 homotrimers induce cancer intrinsic programs to promote proliferation/survival mechanisms, and also influence the tumor immune microenvironment to facilitate immunosuppression. These studies suggested that tumor-promoting oncogenic Col1 produced by cancer cells likely repel T cells and recruit MDSCs.

In addition, the decreased MDSCs and increased T cells in KPPC;Col1^{pdxKO} tumors were also directly associated with unique intratumoral and gut microbiome profiles. These results provide novel insights into the correlation between cancer cell produced collagen and tumor microbiome. The decreased MDSCs and Bacteroidales, coupled with enhanced microaerophilic Campylobacterales in Col1 homotrimer deleted tumors, was accompanied by increased intratumoral T cells, which was abrogated by broad-spectrum antibiotics treatment and decreasing the survival of KPPC;Col1^{pdxKO} mice.

### Methods

Microbial DNA 16S rRNA Gene Sequencing

Fresh tumor samples and fecal samples were collected from KPPC mice, KPPC;Col1^{pdxKO} mice, or wild-type littermate control mice with or without antibiotics treatment. Broad-spectrum antibiotics treatment via oral gavage, as previously described (3), contained vancomycin (50 mg/mL; Sigma-Aldrich), neomycin (10 mg/mL; Sigma-Aldrich), metronidazole (100 mg/mL; Alfa Aesar), and amphotericin (1 mg/mL; X-GEN Pharmaceuticals). During the experiments, mouse drinking water was supplied with ampicillin (1 mg/mL; Sigma-Aldrich), vancomycin (0.5 mg/mL; Sigma-Aldrich), neomycin (0.5 mg/mL; Sigma-Aldrich), metronidazole (1 mg/mL; Alfa Aesar), and amphotericin (0.5 μg/mL; X-GEN Pharmaceuticals). Genomic DNA was isolated with the QIAamp fast DNA stool kit (Qiagen) following the manufacturer's instructions, with an additional step of intensive bead-beating lysis. The V4 region of 16S rRNA gene was amplified by PCR using the 515 forward and 806 reverse primer pairs from 100 ng of purified genomic DNA sample (9). The DNA libraries were purified using the QIAquick gene extraction kit (Qiagen) and sequenced with the Illumina Miseq sequencer system using 2×250 bp paired-end protocol. The paired-end reads were de-multiplexed by QIIME, followed by being merged and de-replicated for chimeras using VSEARCH. UNOISE 3 command algorithm was used as the denoising method of reads (10). Operational taxonomic units (OTUs) were classified with the Silva database version 138 using the Mothur method. For the differential taxa-based univariate analysis, abundant microbiome taxa at Phylum, Class, Order, Family, Genus, Species levels were examined by Mann-Whitney U-test after logit transformation. The detailed computational pipeline for data analysis was based on previous studies (5). Total bacterial DNA content in tumor and fecal samples was examined by qRT-PCR of 16S rRNA.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Vetizou M, Pitt J M, Daillere R, Lepage P. Waldschmitt N, Flament C, et al. Anticancer immunotherapy by CTLA-4 blockade e relies on the gut microbiota. Science 2015;350(6264):1079-84 doi 10.1126/science.aad1329.
2. Gopalakrishnan V, Spencer C N, Nezi L, Reuben A, Andrews M C, Karpinets T V, et al. Gut microbiome modulates response to anti-PD-1 immunotherapy in melanoma patients. Science 2018;359(6371):97-103 doi 10.1126/science.aan4236.
3. Pushalkar S, Hundeyin M, Daley D, Zambirinis C P, Kurz E, Mishra A, et al. The Pancreatic Cancer Microbiome Promotes Oncogenesis by Induction of Innate and Adaptive Immune Suppression. Cancer Discov 2018;8(4):403-16 doi 10.1158/2159-8290.CD-17-1134.
4. Riquelme E, Zhang Y. Zhang L, Montiel M, Zoltan M, Dong W, et al. Tumor Microbiome Diversity and Composition Influence Pancreatic Cancer Outcomes. Cell 2019;178(4):795-806 e12 doi 10.1016/j.cell.2019.07.008.
5. Wang Y. Wiesnoski D H, Helmink B A, Gopalakrishnan V, Choi K, DuPont H L, et al. Fecal microbiota transplantation for refractory immune checkpoint inhibitor-associated colitis. Nature medicine 2018;24(12):1804-8 doi 10.1038/s41591-018-0238-9.
6. Ubeda C. Taur Y, Jenq R R, Equinda M J, Son T, Samstein M, et al. Vancomycin-resistant Enterococcus domination of intestinal microbiota is enabled by antibiotic treatment in mice and precedes bloodstream invasion in humans. The Journal of clinical investigation 2010;120(12):4332-41 doi 10.1172/JCI43918.
7. Toure A M, Landry M, Souchkova O, Kembel S W, Pilon N. Gut microbiota-mediated Gene-Environment interaction in the TashT mouse model of Hirschsprung disease. Sci Rep 2019;9(1):492 doi 10.1038/s41598-018-36967-z.
8. Fessler J, Matson V, Gajewski T F. Exploring the emerging role of the microbiome in cancer immunotherapy. J Immunother Cancer 2019;7(1):108 doi 10.1186/s40425-019-0574-4.
9. Caporaso J G, Lauber C L, Walters W A, Berg-Lyons D, Huntley J, Fierer N, et al. Ultra-high-throughput microbial community analysis on the Illumina HiSeq and MiSeq platforms. ISME J 2012;6(8):1621-4 doi 10.1038/ismej.2012.8.
10. Edgar R C. UNOISE2: improved error-correction for Illumina 16S and ITS amplicon sequencing. bioRxiv 2016:081257 doi 10.1101/081257.

What is claimed is:

1. A method for altering a tumor microbiome comprising administering to a tumor microenvironment an effective amount of an agent that disrupts an interaction between α3β1 integrin and α1 homotrimeric type I collagen.

2. The method of claim 1, wherein the agent is effective to increase an amount of Campylobacterales bacteria in the tumor microbiome.

3. The method of claim 1, wherein the agent is effective to decrease an amount of Bacteriodales bacteria in the tumor microbiome.

4. The method of any of claims 1-3, further comprising administering to the tumor microenvironment an immunotherapeutic.

5. The method of claim 4, wherein the immunotherapeutic comprises a checkpoint blockade therapy.

6. The method of claim 5, wherein the checkpoint blockade therapy comprises an anti-PD-1 antibody.

7. The method of claim 5, wherein the checkpoint blockade therapy comprises an anti-CTLA4 antibody.

8. The method of any of claims 1-7, wherein the agent is an antibody or an antibody fragment that binds to α3β1 integrin.

9. The method of any of claims 1-7, wherein the agent is a chimeric antigen receptor (CAR) polypeptide comprising an antigen binding domain that binds to α3β1 integrin.

10. The method of any one of claims 1-7, wherein the agent comprises a cell comprising a nucleic acid encoding a chimeric antigen receptor (CAR) polypeptide comprising an antigen binding domain that binds to α3β1 integrin.

11. The method of claim 10, wherein the cell is a T cell, a NK cell, an immune cell, an INKT cell, a CD4+ T cell, or a CD8+ T cell.

12. The method of claim 11, wherein the cell is a NK cell.

13. The method of any of claims 1-7, wherein the agent is an antibody or an antibody fragment that binds to α1 homotrimeric type I collagen.

14. The method of any of claims 1-7, wherein the agent is a CAR polypeptide comprising an antigen binding domain that binds to α1 homotrimeric type I collagen.

15. The method of any one of claims 1-7, wherein the agent comprises a cell comprising a nucleic acid encoding a CAR polypeptide comprising an antigen binding domain that binds to al homotrimeric type I collagen.

16. The method of claim 15, wherein the cell is a T cell, a NK cell, an immune cell, an iNKT cell, a CD4+ T cell, or a CD8+ T cell.

17. The method of claim 16, wherein the cell is a NK cell.

18. The method of any of claims 1-14, wherein the tumor microenvironment is a pancreatic tumor microenvironment.

19. The method of any of claims 1-18, further comprising administering an antibiotic to the tumor microenvironment.

20. The method of any of claims 1-19, further comprising isolating bacteria from the tumor microbiome.

21. The method of claim 20, wherein the bacteria is isolated from the tumor microbiome prior to administering the agent.

22. The method of claim 20, wherein the bacteria is isolated from the tumor microbiome after administering the agent.

23. The method of any of claims 1-22, further comprising detecting Campylobacterales bacteria in the tumor microbiome.

24. The method of claim 23, wherein detecting Campylobacterales bacteria comprises sequencing nucleic acid from the tumor microbiome.

25. The method of claim 23, wherein the Campylobacterales bacteria is detected prior to administering the agent.

26. The method of claim 23, wherein the Campylobacterales bacteria is detected after administering the agent.

27. The method of any of claims 1-26, further comprising detecting Bacteriodales bacteria in the tumor microbiome.

28. The method of claim 27, wherein detecting Bacteriodales bacteria comprises sequencing nucleic acid from the tumor microbiome.

29. The method of claim 27, wherein the Bacteriodales bacteria is detected prior to administering the agent.

30. The method of claim 27, wherein the Bacteriodales bacteria is detected after administering the agent.

31. A method of treating a subject for pancreatic cancer, the method comprising:
(a) isolating bacteria from the tumor microbiome; and
(b) administering to the subject an effective amount of:
(i) an agent that disrupts an interaction between α3β1 integrin and α1 homotrimeric type I collagen; and
(ii) an immunotherapy.

32. The method of claim 31, wherein (a) is performed prior to (b).

33. The method of claim 31, wherein (a) is performed subsequent to (b).

34. The method of any of claims 31-33, wherein the agent is effective to increase an amount of Campylobacterales bacteria in the tumor microbiome.

35. The method of any of claims 31-33, wherein the agent is effective to decrease an amount of Bacteriodales bacteria in the tumor microbiome.

36. The method of any of claims 31-35, wherein the agent is an antibody or an antibody fragment that binds to α3β1 integrin.

37. The method of any of claims 31-35, wherein the agent is a chimeric antigen receptor (CAR) polypeptide comprising an antigen binding domain that binds to α3β1 integrin.

38. The method of any one of claims 31-35, wherein the agent comprises a cell comprising a nucleic acid encoding a CAR polypeptide comprising an antigen binding domain that binds to α3β1 integrin.

39. The method of claim 38, wherein the cell is a T cell, a NK cell, an immune cell, an iNKT cell, a CD4+ T cell, or a CD8+ T cell.

40. The method of claim 39, wherein the cell is a NK cell.

41. The method of any of claims 31-35, wherein the agent is an antibody or an antibody fragment that binds to α1 homotrimeric type I collagen.

42. The method of any of claims 31-35, wherein the agent is a CAR polypeptide comprising an antigen binding domain that binds to α1 homotrimeric type I collagen.

43. The method of any one of claims 31-35, wherein the agent comprises a cell comprising a nucleic acid encoding a CAR polypeptide comprising an antigen binding domain that binds to α1 homotrimeric type I collagen.

44. The method of claim 43, wherein the cell is a T cell, a NK cell, an immune cell, an iNKT cell, a CD4+ T cell, or a CD8+ T cell.

45. The method of claim 44, wherein the cell is a NK cell.

46. The method of any of claims 31-42, wherein the immunotherapy comprises a checkpoint blockade therapy.

47. The method of claim 46, wherein the checkpoint blockade therapy comprises an anti-PD-1 antibody.

48. The method of claim 46, wherein the checkpoint blockade therapy comprises an anti-CTLA4 antibody.

49. The method of any of claims 31-48, further comprising detecting Campylobacterales bacteria in the tumor microbiome.

50. The method of claim 49, wherein detecting Campylobacterales bacteria comprises sequencing nucleic acid from the tumor microbiome.

51. The method of claim 49, wherein the Campylobacterales bacteria is detected prior to administering the agent.

52. The method of claim 49, wherein the Campylobacterales bacteria is detected after administering the agent.

53. The method of any of claims 31-52, further comprising detecting Bacteriodales bacteria in the tumor microbiome.

54. The method of claim 53, wherein detecting Bacteriodales bacteria comprises sequencing nucleic acid from the tumor microbiome.

55. The method of claim 53, wherein the Bacteriodales bacteria is detected prior to administering the agent.

56. The method of claim 53, wherein the Bacteriodales bacteria is detected after administering the agent.

57. A method for altering a tumor microbiome comprising administering to a tumor microenvironment an effective amount of an agent that stimulates angiogenesis in the tumor microenvironment.

58. A method for altering a tumor microbiome comprising administering to a tumor microenvironment an effective amount of an agent that inhibits angiogenesis in the tumor microenvironment.

59. A method for altering a tumor microbiome comprising administering to a tumor microenvironment an effective amount of an agent that stimulates epithelial to mesenchymal transition in cells of the tumor microenvironment.

60. A method for altering a tumor microbiome comprising administering to a tumor microenvironment an effective amount of an agent that inhibits epithelial to mesenchymal transition in cells of the tumor microenvironment.

61. A method for altering a tumor microbiome comprising administering to a tumor microenvironment an effective amount of an agent that stimulates pericyte proliferation in the tumor microenvironment.

62. A method for altering a tumor microbiome comprising administering to a tumor microenvironment an effective amount of an agent that inhibits pericyte proliferation in the tumor microenvironment.

63. A method for altering a tumor microbiome comprising administering to a tumor microenvironment an effective amount of an agent that stimulates cellular metabolism in the tumor microenvironment.

64. A method for altering a tumor microbiome comprising administering to a tumor microenvironment an effective amount of an agent that inhibits cellular metabolism in the tumor microenvironment.

65. A method for altering a tumor microbiome comprising administering to a tumor microenvironment an effective amount of an agent that activates fibroblast proliferation in the tumor microenvironment.

66. A method for altering a tumor microbiome comprising administering to a tumor microenvironment an effective amount of an agent that inhibits fibroblast proliferation in the tumor microenvironment.

67. A method for altering a tumor microbiome comprising administering to a tumor microenvironment an effective amount of an agent that degrades extracellular matrix in the tumor microenvironment.

68. The method of claim 67, wherein the agent is a protease.

69. The method of claim 68, wherein the protease is a matrix metalloproteinase.

70. A method for altering a tumor microbiome comprising administering to a tumor microenvironment an effective amount of an agent that stimulates cell-cell adhesion in the tumor microenvironment.

71. A method for altering a tumor microbiome comprising administering to a tumor microenvironment an effective amount of an agent that inhibits cell-cell adhesion in the tumor microenvironment.

72. A method for altering a tumor microbiome comprising administering to a tumor microenvironment an effective amount of an agent that stimulates stromal cell signaling in the tumor microenvironment.

73. A method for altering a tumor microbiome comprising administering to a tumor microenvironment an effective amount of an agent that inhibits stromal cell signaling in the tumor microenvironment.

74. A method for altering a tumor microbiome comprising administering to a tumor microenvironment an effective amount of an agent that disrupts the lymphatic system.

75. A method for altering a tumor microbiome comprising administering to a tumor microenvironment an effective amount of an agent that stimulates lymphangiogenesis.

76. A method for altering a tumor microbiome comprising administering to a tumor microenvironment an effective amount of an agent that inhibits lymphangiogenesis.

77. A method for altering a tumor microbiome comprising administering to a tumor microenvironment an effective amount of an agent that attenuates hypoxia status.

78. A method for altering a tumor microbiome comprising administering to a tumor microenvironment an effective amount of an agent that enhances hypoxia status.

79. A method for altering a tumor microbiome comprising administering to a tumor microenvironment an effective amount of an agent that modifies macrophage polarization.

80. A method for altering a tumor microbiome comprising administering to a tumor microenvironment an effective amount of an agent that modifies macrophage differentiation.

81. The method of any of claims 57-80, wherein the agent is effective to increase an amount of Campylobacterales bacteria in the tumor microbiome.

82. The method of any of claims 57-80, wherein the agent is effective to decrease an amount of Bacteriodales bacteria in the tumor microbiome.

83. The method of any of claims 57-82, wherein the tumor microenvironment is a pancreatic tumor microenvironment.

84. The method of any of claims 57-83, further comprising administering to the tumor microenvironment an immunotherapeutic.

85. The method of claim 84, wherein the immunotherapeutic comprises a checkpoint blockade therapy.

86. The method of claim 85, wherein the checkpoint blockade therapy comprises an anti-PD-1 antibody.

87. The method of claim 85, wherein the checkpoint blockade therapy comprises an anti-CTLA4 antibody.

88. The method of any of claims 57-87, further comprising administering an antibiotic to the tumor microenvironment.

89. The method of any of claims 57-88, further comprising isolating bacteria from the tumor microbiome.

90. The method of claim 89, wherein the bacteria is isolated from the tumor microbiome prior to administering the agent.

91. The method of claim 89, wherein the bacteria is isolated from the tumor microbiome after administering the agent.

92. The method of any of claims 57-91, further comprising detecting Campylobacterales bacteria in the tumor microbiome.

93. The method of claim 92, wherein detecting Campylobacterales bacteria comprises sequencing nucleic acid from the tumor microbiome.

94. The method of claim 92, wherein the Campylobacterales bacteria is detected prior to administering the agent.

95. The method of claim 92, wherein the Campylobacterales bacteria is detected after administering the agent.

96. The method of any of claims 57-95, further comprising detecting Bacteriodales bacteria in the tumor microbiome.

97. The method of claim 96, wherein detecting Bacteriodales bacteria comprises sequencing nucleic acid from the tumor microbiome.

98. The method of claim 96, wherein the Bacteriodales bacteria is detected prior to administering the agent.

99. The method of claim 96, wherein the Bacteriodales bacteria is detected after administering the agent.
